(19) United States
(12) Patent Application Publication
(10) Pub. No.: US 2012/0207853 A1
(43) Pub. Date: Aug. 16, 2012

(54) Methods of treating or preventing influenza associated illness with oxidative reductive potential water solutions

(75) Inventors: Hojabr Alimi, Petaluma, CA (US); Eileen Thatcher, Petaluma, CA (US)

(73) Assignee: OCULUS INNOVATIVE SCIENCES, INC., Petaluma, CA (US)

(21) Appl. No.: 13/320,225

(22) PCT Filed: May 10, 2010
(86) PCT No.: PCT/US10/34238

§ 371 Date: May 2, 2012

Related U.S. Application Data
(60) Provisional application No. 61/177,275, filed on May 11, 2009.

(51) Int. Cl. A61K 33/20; A01P 1/00; A61P 31/16; A01N 59/00; A61K 33/40; A61P 31/12

(52) U.S. Cl. 424/616; 424/663; 424/661; 424/665

(57) ABSTRACT

The present invention provides methods of treating, reducing, and/or preventing the incidence of an influenza related viral infection in a patient comprising administering a therapeutically effective amount of an oxidative reductive potential (ORP) water solution. The present invention also provides methods of reducing or preventing the incidence of an influenza related viral infection in a patient associated with a medical device comprising contacting the medical device with an effective amount of an ORP water solution.

### BACKGROUND OF THE INVENTION

Influenza, commonly referred to as the flu, is an infectious respiratory illness caused by RNA viruses of the family Orthomyxoviridae. Influenza affects birds and mammals causing mild to severe illness and, at times, can lead to death. The most common symptoms of the illness are chills, fever, sore throat, muscle pains, severe headache, coughing, weakness and general discomfort. Upon infection, young children, the elderly, and people with certain health conditions (e.g., asthma, diabetes, heart disease, etc.) are often at high risk of developing serious influenza complications such as bacterial pneumonia, ear infections, sinus infections, dehydration, and/or worsening of preexisting chronic medical conditions.

In humans, influenza viruses are most commonly thought to spread from person to person through coughing or sneezing by those infected with a virus. Influenza can be transmitted by bird droppings, saliva, nasal secretions, feces and blood. It is also possible to become infected by physical contact with an object (e.g., door knob, faucet, etc.) having the influenza virus on its surface. Influenza viruses are highly contagious and a healthy adult can often infect others before symptoms of the illness even develop. According to the U.S. Centers for Disease Control and Prevention (CDC) and the World Health Organization (WHO), the single best way to prevent the influenza virus is to get a vaccination each year. The most common vaccinations include the “flu shot,” an inactivated vaccine (containing killed virus) that is given with a needle, and a nasal-spray, a vaccine made with live, weakened influenza viruses. Vaccination enables development of antibodies which protect against influenza virus infection.

However, in this regard, the world's influenza vaccine production capacity is rather limited because the vaccine virus must be grown in chicken eggs and cultured before it is processed. Thus, current production methods are limited in the number of vaccine doses which can be produced in a year and, further, are difficult to expand quickly in the case of an influenza pandemic. In addition, influenza viruses rapidly evolve and new strains quickly replace the older ones. As such, a vaccine formulated for one year may be ineffective in the following year. Furthermore, manufacturing a specialized influenza vaccine for a new variant (e.g., “Bird Flu,” “Swine Flu,” etc.) would come at the expense of seasonal vaccine production and might lead to higher infection rates (and, possibly, mortality rates) associated with normal seasonal strains.

Accordingly, the need exists for new antiviral agents and methods of their use in the treatment and prevention of influenza. The present invention provides such methods. These and other advantages of the invention, as well as additional inventive features, will be apparent from the description of the invention provided herein.

### BRIEF SUMMARY OF THE INVENTION

The present invention provides a method of treating, reducing, and/or preventing the incidence of a viral infection in a patient comprising administering a therapeutically effective amount of an oxidative reductive potential (ORP) water solution to an infectious virus present on the surface of a tissue, wherein the infectious virus is an influenza virus and the tissue is selected from the group consisting of lower and upper respiratory tract tissue, corneal tissue, inner ear tissue, urinary tract tissue, mucosa tissue, dental tissue and synovial tissue.

The invention also provides a method of reducing or preventing the incidence of a viral infection in a patient associated with a medical device comprising contacting the medical device with an effective amount of an ORP water solution, wherein an influenza virus is present on the surface of the device.

### BRIEF DESCRIPTION OF THE DRAWINGS

FIG. 1 illustrates a three-chambered electrolysis cell for producing an exemplary ORP water solution administered in accordance with the invention.

FIG. 2 illustrates a three-chambered electrolysis cell and depicts ionic species that are believed to be generated during the production of an exemplary ORP water solution administered in accordance with the invention.

FIG. 3 is a schematic flow diagram of a process for producing an exemplary ORP water solution administered in accordance with the invention.

### DETAILED DESCRIPTION OF THE INVENTION

The present invention provides a method of treating, reducing, and/or preventing the incidence of influenza related viral infections in a patient comprising administering to the patient a therapeutically effective amount of an oxidative reductive potential (ORP) water solution (also known as super-oxidized water (SOW)). The term “treating,” as used herein means affecting a cure, reducing the infectious micro-organism population and/or ameliorating the signs and symptoms of a condition or disease.

The method of the present invention can also be used for reducing or preventing (e.g., inhibiting the onset of, inhibiting the escalation of, decreasing the likelihood of) acute and chronic influenza related viral infections. More specifically the phrase “reducing or preventing the incidence of a viral infection,” as used herein, is meant to convey that the incidence of infection is decreased by at least about 5%, preferably by at least about 10%, more preferably by at least about 20%, even more preferably by at least about 25%, even more preferably by at least about 50%, even more preferably by at least about 2-fold, even more preferably by at least about 5-fold, even more preferably by at least about 10-fold, even more preferably by at least about 100-fold even more preferably by at least about 100-fold, and most preferably by at least about 1.000-fold.

The term “a therapeutically effective amount,” as used herein, refers to an amount that is adequate (sufficient) to treat a disease or condition such as, e.g., an influenza related infection or colonization.

By “contacting the medical device with an effective amount,” as used herein, it is meant bringing an adequate (sufficient) amount into a close enough physical proximity to effect the goals of the claim.

As used herein, “patient” is any suitable patient which can be infected with or be a host for an influenza virus. In one embodiment, the patient is a bird or a mammal. In a preferred embodiment, the patient is a human.

The present invention provides a method of treating, reducing, and/or preventing the incidence of influenza related viral infections. The influenza virus can be influenza A, influenza B, or influenza C. In a preferred embodiment the present invention is useful for the treatment, reduction, and/or prevention of influenza A. Influenza A virus strains are categorized according to two proteins found on the surface of the virus: hemagglutinin (H) and neuraminidase (N). All influenza A viruses contain hemagglutinin and neuraminidase, but the structure of these proteins differs from strain to strain due to rapid genetic mutation in the viral genome. Accordingly, the present invention is useful for the treatment, reduction, and/or prevention of infections related to all subtypes of influenza A. Exemplary subtypes of influenza A include, but are not limited to: H1N1, H1N2, H2N2, H3N1, H3N2, H3N8, H5N1, H5N2, H5N3, H5N8, H5N9, H7N1, H7N2, H7N3, H7N4, H7N7, H9N2, and H10N7. In a preferred embodiment, the method of the present invention is employed to treat, reduce, and/or prevent the incidence of viral infections related to H1N1 or H3N1 strains of influenza A.

The ORP water solution administered in accordance with the invention can be acidic, neutral or basic, and generally can have a pH of from about 1 to about 14. Within this pH range, the ORP water solution can be safely applied in suitable quantities, e.g., to surfaces without damaging the surfaces or harming objects, such as human skin, that comes into contact with the ORP water solution. Preferably, the pH of the ORP water solution administered in accordance with the invention is from about 6.0 to about 8.0. More preferably, the pH of the ORP water solution is from about 6.4 to about 7.8, and still more preferably, the pH is from about 7.4 to about 7.6.

The ORP water solution administered in accordance with the invention can have an oxidation-reduction potential of from about −400 millivolts (mV) to about +1300 millivolts (mV). This potential is a measure of the tendency (i.e., the potential) of a solution to either accept or transfer electrons that are sensed by a metal electrode and compared with a reference electrode in the same solution. This potential may be measured by standard techniques including, for example, measuring the electrical potential in millivolts of the ORP water solution relative to standard reference such as, e.g., a silver/silver chloride electrode.

The ORP water solution administered in accordance with the invention preferably has a potential of from about −400 mV to about +1300 mV. More preferably, the ORP water solution has a potential of from about 0 mV to about +1250 mV, and still more preferably from about +500 mV to about +1250 mV. Even more preferably, the ORP water solution administered in accordance with the present invention has a potential of from about +800 mV to about +1100 mV, and most preferably from about +800 mV to about +1000 mV.

Various ionic and other species may be present in the ORP water solution administered in accordance with the invention. For example, the ORP water solution may contain chlorine (e.g., free chlorine and bound chlorine), and dissolved oxygen and, optionally, ozone and peroxides (e.g., hydrogen peroxide). The presence of one or more of these species is believed to contribute to at least the disinfectant ability of the ORP water solution to kill a variety of microorganisms, such as influenza viruses.

The free chlorine species can include one or more species selected from the group consisting of hypochlorous acid (HOCl), hypochlorite ions (OCl⁻), and sodium hypochlorite (NaOCl), chloride ion (Cl⁻), and optionally, chlorine dioxide (ClO₂), dissolved chlorine gas (Cl₂), precursors thereof and mixtures thereof. In a preferred embodiment, the ORP water solution administered in accordance with the invention comprises free chlorine including, but is not limited to, hypochlorous acid (HClO), hypochlorite ions (ClO⁻), sodium hypochlorite (NaOCl), and precursors thereof. The ratio of hypochlorous acid to hypochlorite ion is dependent upon pH. At a pH of 7.4, hypochlorous acid levels are typically from about 25 ppm to about 75 ppm. Temperature also impacts the ratio of the free chlorine component.

Bound chlorine typically includes chlorine in chemical combination with, e.g., ammonia or organic amines (e.g., chloramines). Bound chlorine is preferably present in an amount of up to about 20 ppm.

One or more chlorine species, one or more additional superoxidized water species (e.g., one or more additional oxidizing species such as, e.g., oxygen) can be present in the ORP water solution administered in accordance with the invention in any suitable amount. The levels of these components may be measured by any suitable method, including methods known in the art.

The total amount of free chlorine species is preferably from about 10 ppm to about 400 ppm, more preferably from about 50 ppm to about 200 ppm, and most preferably from about 50 ppm to about 80 ppm. The amount of hypochlorous acid is preferably from about 15 ppm to about 35 ppm. The amount of sodium hypochlorite is preferably in the range of from about 25 ppm to about 50 ppm. Optionally, chlorine dioxide levels are preferably less than about 5 ppm.

The chlorine content may be measured by methods known in the art, such as the DPD colorimeter method (Lamotte Company, Chestertown, Md.) or other known methods such as, e.g., methods established by the Environmental Protection Agency. In the DPD colorimeter method, a yellow color is formed by the reaction of free chlorine with N,N-diethyl-p-phenylenediamine (DPD) and the intensity is measured with a calibrated calorimeter that provides the output in parts per million. Further addition of potassium iodide turns the solution a pink color to provide the total chlorine value. The amount of bound chlorine present is then determined by subtracting free chlorine from the total chlorine. The total amount of oxidizing chemical species present in the ORP water solution is preferably in the range of about 2 millimolar (mM), which includes the aforementioned chlorine species, oxygen species, and additional species, including those, which can be difficult to measure such as, e.g., Cl⁻, ClO₃, Cl₂⁻, and ClOₓ.

In one embodiment, the ORP water solution administered in accordance with the invention comprises one or more chlorine species and one or more additional superoxidized water species (e.g., one or more additional oxidizing species such as, e.g., oxygen). Preferably, the chlorine species present is a free chlorine species as set forth above.

In one embodiment, the ORP water solution includes one or more chlorine species or one or more precursors thereof, and one or more additional superoxidized water species or one or more precursors thereof, and, optionally, hydrogen peroxide.

In yet another embodiment, the ORP water solution administered in accordance with the invention includes one or more chlorine species (e.g., hypochlorous acid and sodium hypochlorite) or one or more precursors thereof and one or one or more additional superoxidized water species (e.g., one or more oxygen species, dissolved oxygen) or one or more precursors thereof and has a pH of from about 6 to about 8. More preferably from about 6.2 to about 7.8, and most preferably from about 7.4 to about 7.6. An exemplary ORP water solution administered in accordance with the present invention can comprise, e.g., from about 15 ppm to about 35 ppm hypochlorous acid, from about 25 ppm to about 50 ppm sodium hypochlorite, from about 1 ppm to about 4 ppm of one or more additional superoxidized water species and a pH of from about 6.2 to about 7.8.

In another embodiment, the ORP water solutions administered in accordance with the invention comprise one or more oxidized water species which can yield free radicals (such as, e.g., hydroxyl radicals) on exposure to iron. The ORP water can optionally include one or more chemical compounds generated during the production thereof such as, e.g., sodium hydroxide (NaOH), chlorine dioxide (ClO₂), peroxides (e.g., hydrogen peroxide (H₂O₂), and ozone (O₃) although, it has been reported that sodium hydroxide, chlorine dioxide, hydrogen peroxide, and ozone may react with hypochlorite resulting in their consumption and the production of other chemical species.

While in no way limiting the present invention, it is believed that the control of pH and other variables (e.g., salinity) can provide stable ORP water solutions, which contain one or more chlorine species or precursors thereof, such as, e.g., hypochlorous acid and hypochlorite ions, and one or more additional superoxidized water species (e.g., oxygen) or one or more precursors thereof.

Accordingly, combinations of these factors can characterize the ORP water for use in accordance with the invention, for example, the wherein the pH of the ORP water is from about 6.0 to about 8.0 and the concentration of free chlorine species in the ORP water is from about 10 ppm to about 400 ppm.

It has been found that the ORP water solution administered in accordance with the invention is virtually free of toxicity to normal tissues and normal mammalian cells. The ORP water solution administered in accordance with the invention causes no significant decrease in the viability of eukaryotic cells, no significant increase in apoptosis, no significant acceleration of cell aging and/or no significant oxidative DNA damage in mammalian cells. The non-toxicity is particularly advantageous, and perhaps even surprising, given that the disinfecting power of the ORP water solution administered in accordance with the invention is roughly equivalent to that of hydrogen peroxide, yet is significantly less toxic than hydrogen peroxide is to normal tissues and normal mammalian cells. These findings demonstrate that the ORP water solution administered in accordance with the present invention is safe for use, e.g., in mammals, including humans.

For the ORP water solution administered in accordance with the invention, the cell viability rate is preferably at least about 65%, more preferably at least about 70%, and still more preferably at least about 75% after an about 30 minute exposure to the ORP water solution. In addition, the ORP water solution administered in accordance with the invention preferably causes only up to about 10% of cells, more preferably only up to about 5% of cells, and still more preferably only up to about 3% of cells to expose Annexin-V on their cellular surfaces when contacted with the ORP water solution for up to about thirty minutes or less (e.g., after about thirty minutes or after about five minutes of contact with the ORP water solution).

Further, the ORP water solution administered in accordance with the invention preferably causes less than about 15% of cells, more preferably less than about 10% of cells, and still more preferably less than about 5% of cells to express the SA-β-galactosidase enzyme after chronic exposure to the OPR water solution. The ORP water solution administered in accordance with the invention preferably causes caused the same fraction of the oxidative DNA adduct formation caused by saline solution, e.g., less than about 20% of the oxidative DNA adduct formation, less than about 10% of the oxidative DNA adduct formation, or about 5% or less of the oxidative DNA adduct formation normally caused by hydrogen peroxide in cells treated under equivalent conditions.

The ORP water solution administered in accordance with the invention produces no significant RNA degradation. Accordingly, RNA extracted from human cell cultures after an about 30 minutes exposure to the ORP water solution or r at about 3 hours after an about 30 minute-exposure, and analyzed by denaturing gel electrophoresis, will typically show no significant RNA degradation and will typically exhibit two discreet bands corresponding to the ribosomal eukaryotic RNAs (i.e. 28S and 18S) indicating that the ORP water solution administered in accordance with the invention leaves the RNA substantially intact. Similarly, RNA extracted from human cell cultures after about 30 minutes of exposure to the ORP water solution or after about 3 hours of exposure, can be subjected reverse transcription and amplification (RT-PCR) of the constitutive human GAPDH (Glyceraldehyde-3-phosphate dehydrogenase) gene and result in a strong GAPDH band on gel electrophoresis of the RT-PCR products. By contrast, cells treated with HP for a similar period show significant RNA degradation and little if any GAPDH RT-PCR product.

In accordance with the toxicity profile of the ORP water solution discussed above, the present invention provides a method of administering an ORP water solution to the surface of a tissue of the patient. The tissue can be any tissue which can be infected with or be a host for an influenza virus. In one embodiment, the tissue is selected from the group consisting of lower and upper respiratory tract tissue, corneal tissue, inner ear tissue, urinary tract tissue, mucosa tissue, dental tissue, and synovial tissue. In a preferred embodiment, the ORP water solution is administered to lower respiratory tract tissue or upper respiratory tract tissue.

The ORP water solution used in accordance with the present invention can be administered using any suitable method of administration known in the art. For instance, the ORP water solution can be administered parenterally, endoscopically or directly to the surface of any affected biological tissue, e.g., to the skin and/or one or more mucosal surfaces. Parenteral administration can include using, for example, administering the ORP water solution intramuscularly, subcutaneously, intravenously, intra-arterially, intrathecally, intravesically or into a synovial space. Endoscopic administration of the ORP water solution can include using, e.g., bronchoscopy, colonoscopy, sigmoidoscopy, hysterscopy, laproscopy, athroscopy, gastroscopy or a transurethral approach. Administering the ORP water solution to a mucosal surface can include, e.g., administration to a nasal, oral, tracheal, bronchial, esophageal, gastric, intestinal, peritoneal, urethral, vesicular, urethral, vaginal, uterine, fallopian, and synovial mucosal surface. Parenteral administration also can include administering the ORP water solution used in accordance with the invention intravenously, subcutaneously, intramuscularly, or intraperitoneally.

The ORP water solution used in accordance with the invention can be administered topically, e.g., as a liquid, spray, mist, aerosol or steam by any suitable process, e.g., by aerosolization, nebulization or atomization. The ORP solution of the present invention can be administered to the upper airway as a steam or a spray. When the ORP water solution is administered by aerosolization, nebulization or atomization, it is preferably administered in the form of droplets having a diameter in the range of from about 0.1 micron to about 100 microns, preferably from about 1 micron to about 10 microns. In one embodiment, the method of the present invention includes administering the ORP water solution in the form of droplets having a diameter in the range of from about 1 micron to about 10 microns to one or more mucosal tissues, e.g., one or more upper respiratory tissues and/or lung tissues.

Methods and devices, which are useful for aerosolization, nebulization and atomization, are well known in the art. Medical nebulizers, for example, have been used to deliver a metered dose of a physiologically active liquid into an inspiration gas stream for inhalation by a recipient. See, e.g., U.S. Pat. No. 6,598,602. Medical nebulizers can operate to generate liquid droplets, which form an aerosol with the inspiration gas. In other circumstances medical nebulizers may be used to inject water droplets into an inspiration gas stream to provide gas with a suitable moisture content to a recipient, which is particularly useful where the inspiration gas stream is provided by a mechanical breathing aid such as a respirator, ventilator or anesthetic delivery system.

An exemplary nebulizer is described, for example, in WO 95/01137, which describes a hand held device that operates to eject droplets of a medical liquid into a passing air stream (inspiration gas stream), which is generated by a recipient's inhalation through a mouthpiece. Another example can be found in U.S. Pat. No. 5,388,571, which describes a positive-pressure ventilator system which provides control and augmentation of breathing for a patient with respiratory insufficiency and which includes a nebulizer for delivering particles of liquid medication into the airways and alveoli of the lungs of a patient. U.S. Pat. No. 5,312,281 describes an ultrasonic wave nebulizer, which atomizes water or liquid at low temperature and reportedly can adjust the size of mist. In addition, U.S. Pat. No. 5,287,847 describes a pneumatic nebulizing apparatus with scalable flow rates and output volumes for delivering a medicinal aerosol to neonates, children and adults. Further, U.S. Pat. No. 5,063,922 describes an ultrasonic atomizer. The ORP water solution also may be dispensed in aerosol form as part of an inhaler system for treatment of infections in the lungs and/or air passages or for the healing of wounds in such parts of the body.

For larger scale applications, a suitable device may be used to disperse the ORP water solution into the air including, but not limited to, humidifiers, misters, foggers, vaporizers, atomizers, water sprays, and other spray devices. Such devices permit the dispensing of the ORP water solution on a continuous basis. An ejector which directly mixes air and water in a nozzle may be employed. The ORP water solution may be converted to steam, such as low pressure steam, and released into the air stream. Various types of humidifiers may be used such as ultrasonic humidifiers, stream humidifiers or vaporizers, and evaporative humidifiers. The particular device used to disperse the ORP water solution may be incorporated into a ventilation system to provide for widespread application of the ORP water solution throughout an entire house or healthcare facility (e.g., hospital, nursing home, etc.).

In accordance with the invention, the ORP water solution can be administered alone or in combination with one or more pharmaceutically acceptable carriers, e.g., vehicles, adjuvants, excipients, diluents, combinations thereof, and the like, which are preferably compatible with one or more of the species that exist in the ORP water solution. One skilled in the art can easily determine the appropriate formulation and method for administering the ORP water solution used in accordance with the present invention. Any necessary adjustments in dose can be readily made by a skilled practitioner to address the nature and/or severity of the condition being treated in view of one or more clinically relevant factors, such as, e.g., side effects, changes in the patient's overall condition, and the like.

For example, the ORP water solution can be formulated by combining or diluting the ORP water solution with up to about 25% (wt./wt. or vol./vol.) of a suitable carrier, up to about 50% (wt./wt. or vol./vol.) of a suitable carrier, up to about 75% (wt./wt. or vol./vol.) of a suitable carrier, up to about 90% (wt./wt. or vol./vol.) of a suitable carrier, up to about 95% (wt./wt. or vol./vol.) of a suitable carrier, or even with up to about 99% (wt./wt. or vol./vol.) or more of a suitable carrier. Suitable carriers can include, e.g., water (e.g., distilled water, sterile water, e.g., sterile water for injection, sterile saline and the like). Suitable carriers also can include one or more carriers described in U.S. patent application Ser. No. 10/916,278. Exemplary formulations can include, e.g., solutions in which the ORP water solution is diluted with sterile water or sterile saline, wherein the ORP water solution is diluted by up to about 25% (vol./vol.), by up to about 50% (vol./vol.), by up to about 75% (vol./vol.), by up to about 90% (vol./vol.), by up to about 95% (vol./vol.), or by up to 99% (vol./vol.) or more of a suitable carrier.

The ORP water solution administered in accordance with the invention can further be combined with (or be administered in conjunction with) one or more additional therapeutic agents, e.g., one or more active compounds selected from the group consisting of antibacterial agents (e.g., antibiotics), anti-viral agents, anti-inflammatory agents, and combinations thereof.

The therapeutically effective amount administered to the patient, e.g., a mammal, particularly a human, in the context of the present invention should be sufficient to affect a therapeutic or prophylactic response in the patient over a reasonable time frame. The dose can be readily determined using methods that are well known in the art. One skilled in the art will recognize that the specific dosage level for any particular patient will depend upon a variety of potentially therapeutically relevant factors. For example, the dose can be determined based on the strength of the particular ORP water solution employed, the severity of the condition, the body weight of the patient, the age of the patient, the physical and mental condition of the patient, general health, sex, diet, the frequency of applications, and the like. The size of the dose also can be determined based on the existence, nature, and extent of any adverse side effects that might accompany the administration of a particular ORP water solution. It is desirable, whenever possible, to keep adverse side effects to a minimum.

Factors, which can be taken into account for a specific dosage can include, for example, bioavailability, metabolic profile, time of administration, route of administration, rate of excretion, the pharmacodynamics associated with a particular ORP water solution in a particular patient, and the like. Other factors can include, e.g., the potency or effectiveness of the ORP water solution with respect to the particular condition to be treated, the severity of the symptoms presented prior to or during the course of therapy, and the like. In some instances, what constitutes a therapeutically effective amount also can be determined, in part, by the use of one or more of the assays, e.g., bioassays, which are reasonably clinically predictive of the efficacy of a particular ORP water solution for the treatment or prevention of a particular condition.

One skilled in the art will appreciate that suitable methods of administering the ORP water solution used in accordance with the present invention are available, and, although more than one route of administration can be used, a particular route can provide a more immediate and more effective reaction than another route. The therapeutically effective amount can be the dose necessary to achieve an “effective level” of the ORP water solution in an individual patient, independent of the number of applications a day. The therapeutically effective amount can be defined, for example, as the amount required to be administered to an individual patient to achieve a blood level, tissue level, and/or intracellular level of the ORP water solution (or one or more active species contained therein) to prevent or treat the condition in the patient.

When the effective level is used as a preferred endpoint for dosing, the actual dose and schedule can vary depending, for example, upon inter-individual differences in pharmacokinetics, distribution, metabolism, and the like. The effective level also can vary when the ORP water solution is used in combination with one or more additional therapeutic agents, e.g., one or more anti-infective agents, one or more “moderating,” “modulating” or “neutralizing agents,” e.g., as described in U.S. Pat. Nos. 5,334,383 and 5,622,848, one or more anti-inflammatory agents, and the like.

An appropriate indicator can be used for determining and/or monitoring the effective level. For example, the effective level can be determined by direct analysis (e.g., analytical chemistry) or by indirect analysis (e.g., with clinical chemistry indicators) of appropriate patient samples (e.g., blood and/or tissues). The effective level also can be determined, for example, by direct or indirect observations such as, e.g., the concentration of urinary metabolites, changes in markers associated with the condition (e.g., viral count in the case of a viral infection), histopathology and immunochemistry analysis, positive changes in image analysis (e.g. X ray, CT scan, NMR, PET, etc), nuclear medicine studies, decrease in the symptoms associated with the conditions, and the like.

Methods in accordance with the invention include the sterilization of and reduction in the incidence of viral infections associated with medical devices by contacting the devises with an effective amount of an oxidative reductive potential (ORP) water solution. Such devices include, but are not limited to, epicardial leads, cardiac and cerebral pacemakers, defibrillators, left ventricular assist devices, mechanical heart valves, total artificial hearts, ventriculoatrial shunts, pledgets, patent ductus arteriosus occlusion devices (plugs, double umbrellas, buttons, discs, embolization coils), atrial septal defect and ventricular septal defect closure devices (bard clamshell occluders, discs, buttons, double umbrellas), conduits, patches, peripheral vascular stents, coronary artery stents, vascular grafts, abdominal mesh reinforcements, hemodialysis shunts, intra-aortic balloon pumps, angioplasty balloon catheters, angiography catheters, vena caval filters, endotracheal tubes, cochlear implants, tympanostomy tubes, bioabsorbable osteoconductive drug-releasing hard tissue fixation devices, artificial joint replacements and other orthopedic implants. Further, methods in accordance with the invention can be used to sterilize or reduce the incidence of viral infections associated with medical devices that are not fully implanted such as, e.g., contact lenses, intrauterine devices, dental and orthodontic appliances and fixtures, urinary catheters, intravenous catheters, sutures and surgical staples.

The ORP water solution may be dispensed, impregnated, coated, covered or otherwise applied to the medical device by any suitable method. For example, individual portions of medical device may be treated with a discrete amount of the ORP water solution. The medical device or its components may be dipped in, soaked in or sprayed with the ORP water solution. The ORP water may be contacted, come into physical contact with, the medical device for any suitable time period provided that the contact results in an at least about a 3 log reduction in viral concentration, preferably an at least about a 3.5 log reduction in viral concentration, more preferably an at least about a 4 log reduction in viral concentration, even more preferably an at least about a 5 log reduction in viral concentration, and most preferably an at least about a 6 log reduction in viral concentration. In certain embodiments, the ORP water solution can provide at least about a 7 log reduction in viral concentration.

Accordingly, suitable contact times include at least about 10 seconds, at least about 30 seconds, at least 1 minute, at least about 5 minutes, at least about 10 minutes, at least about 20 minutes, at least about 30 minutes, at least about 1 hour, at least about 2 hours, at least about 3 hours, at least about 4 hours, at least about 12 hour, at least about 24 hour, at least about 2 days, at least about 3 days, at least about 5 days, and about 1 week.

The ORP water solution used to contact the medical device can be at any suitable temperature including, e.g., room temperature, 37° C. or >100° C. Further, the ORP water used to contact the medical device may also be comprised of any suitable antimicrobial, including, e.g., bleaches, antifungals, antibiotics, antivirals, disinfectant salts, alcohols or biologics.

If the medical device has a web structure, a mass treatment of a continuous web of medical device material with the ORP water solution is carried out. The entire web of medical device material may be soaked in the ORP water solution. Alternatively, as the medical device web is spooled, or even during creation of a nonwoven substrate, the ORP water solution is sprayed or metered onto the web.

For small scale applications, the ORP water solution may be dispensed through a spray bottle that includes a standpipe and pump. Alternatively, the ORP water solution may be packaged in aerosol containers. Aerosol containers generally include the product to be dispensed, propellant, container, and valve. The valve includes both an actuator and dip tube. The contents of the container are dispensed by pressing down on the actuator. The various components of the aerosol container are compatible with the ORP water solution. Suitable propellants may include a liquefied halocarbon, hydrocarbon, or halocarbon-hydrocarbon blend, or a compressed gas such as carbon dioxide, nitrogen, or nitrous oxide. Aerosol systems typically yield droplets that range in size from about 0.15 μm to about 5 μm.

Conventional ORP water solutions have an extremely limited shelf-life, usually only a few hours. As a result of this short lifespan, using conventional ORP water solutions requires the production to take place in close proximity to the point of use. From a practical standpoint, this means that the facility, e.g., a healthcare facility such as a hospital, must purchase, house and maintain the equipment necessary to produce conventional ORP water solution. Additionally, conventional manufacturing techniques have not been able to produce sufficient commercial-scale quantities to permit widespread use, e.g., as a general disinfecting agent for healthcare facilities.

Unlike conventional ORP water solutions, the ORP water solution administered in accordance with the invention is stable for at least about twenty-hours after its preparation. In addition, the ORP water solution administered in accordance with the invention is generally environmentally safe and, thus, avoids the need for costly disposal procedures. Preferably, the ORP water solution administered in accordance with the invention is stable for at least about one week (e.g., one week, two weeks, three weeks, four weeks or more), and more preferably at least about two months. Still more preferably, the ORP water solution administered in accordance with the invention is stable for at least about six months. Even more preferably, the ORP water solution administered in accordance with the invention is stable for at least about one year, and most preferably is stable for more than about one year, e.g., at least about two years or at least about three years.

Stability can be measured based on the ability of the ORP water solution to remain suitable for one or more uses, for example, decontamination, disinfection, sterilization, anti-microbial cleansing, and wound cleansing, for a specified period of time after its preparation under normal storage conditions (e.g., room temperature). The stability of the ORP water solution administered in accordance with the invention also can be measured by storage under accelerated conditions, e.g., from about 30° C. to about 60° C., in which the ORP water solution preferably is stable for up to about 90 days, and more preferably for up to about 180 days.

Stability also can be measured based on the concentration over time of one or more species (or precursors thereof) present in solution during the shelf-life of the ORP water solution. Preferably, the concentrations of one or more species, e.g., free chlorine, hypochlorous acid and one or more additional superoxidized water species and are maintained at about 70% or greater of their initial concentration for at least about two months after preparation of the ORP water solution. More preferably, the concentration of one of more of these species is maintained at about 80% or greater of their initial concentration for at least about two months after preparation of the ORP water solution. Still more preferably, the concentration of one or more of such species is maintained at about 90% or greater, and most preferably is maintained at about 95% or greater, of their initial concentration for at least about two months after preparation of the ORP water solution.

Stability also can be determined based on the reduction in the amount of organisms present in a sample following exposure to the ORP water solution. Measuring the reduction of organism concentration can be made on the basis of any suitable organism including, e.g., bacteria, fungi, yeasts, or viruses. Suitable organisms can include, e.g., Escherichia coli, Staphylococcus aureus, Candida albicans, and Bacillus athrophaeus (formerly B. subtilis).

The ORP water solution administered in accordance with the present invention can be produced by an oxidation-reduction process, e.g., by an electrolytic process or redox reaction, in which electrical energy is used to produce one or more chemical changes in an aqueous solution. Exemplary processes for preparing suitable ORP water solutions are described, e.g., in U.S. Patent Application Publication Nos. US 2005/0139808 and US 2005/0142157.

In the electrolytic process, electrical energy is introduced into and transported through water by the conduction of electrical charge from one point to another in the form of an electrical current. In order for the electrical current to arise and subsist there should be charge carriers in the water, and there should be a force that makes the carriers move. The charge carriers can be electrons, as in the case of metal and semiconductors, or they can be positive and negative ions in the case of solutions. A reduction reaction occurs at the cathode while an oxidation reaction occurs at the anode. At least some of the reductive and oxidative reactions that are believed to occur are described in International Application WO 03/048421 A1.

As used herein, water produced at an anode is referred to as anode water and water produced at a cathode is referred to as cathode water. Anode water typically contains oxidized species produced from the electrolytic reaction while cathode water typically contains reduced species from the reaction. Anode water generally has a low pH, typically of from about 1 to about 6.8. The anode water preferably contains chlorine in various forms including, for example, chlorine gas, chloride ions, hydrochloric acid and/or hypochlorous acid, or one or more precursors thereof. Oxygen in various forms is also preferably present including, for example, oxygen gas, and possibly one or more species formed during production (e.g., peroxides, and/or ozone), or one or more precursors thereof. Cathode water generally has a high pH, typically from about 7.2 to about 11. Cathode water can contain hydrogen gas, hydroxyl radicals, and/or sodium ions.

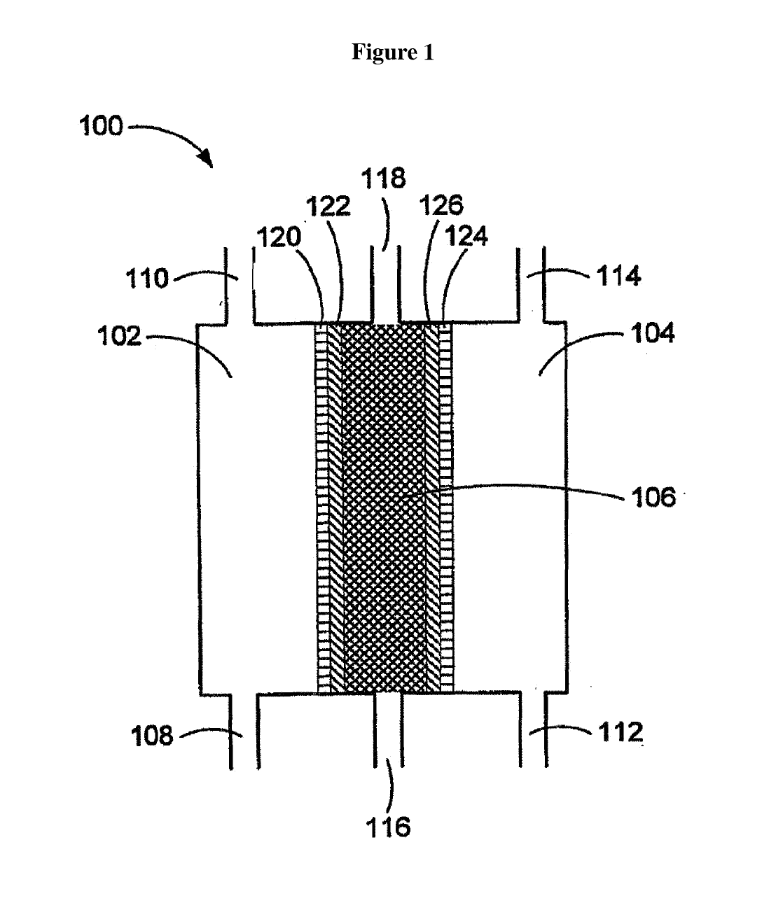

The ORP water solution administered in accordance with the invention can include a mixture of anode water (e.g., water produced in the anode chamber of an electrolytic cell) and cathode water (e.g., water produced in the cathode chamber of an electrolysis cell). Preferably, the ORP water solution administered in accordance with the present invention contains cathode water, e.g., in an amount of from about 10% by volume to about 90% by volume of the solution. More preferably, cathode water is present in the ORP water solution in an amount of from about 10% by volume to about 50% by volume, and still more preferably of from about 20% by volume to about 40% by volume of the solution, e.g., from about 20% by volume to about 30% by volume of the solution. Additionally, anode water can be present in the ORP water solution, e.g., in an amount of from about 50% by volume to about 90% by volume of the solution. Exemplary ORP water solutions can contain from about 10% by volume to about 50% by volume of cathode water and from about 50% by volume to about 90% by volume of anode water. The anode and cathode water can be produced using the three-chambered electrolysis cell shown in FIG. 1.

The ORP water solution administered in accordance with the invention is preferably produced using at least one electrolysis cell comprising an anode chamber, a cathode chamber and a salt solution chamber located between the anode and cathode chambers, wherein at least some of the anode and cathode water are combined such that the ORP water solution comprises anode water and cathode water. A diagram of an exemplary three chamber electrolysis cell that can be used in preparing an exemplary ORP water solution is shown in FIG. 1.

The electrolysis cell 100 has an anode chamber 102, cathode chamber 104 and salt solution chamber 106. The salt solution chamber is located between the anode chamber 102 and cathode chamber 104. The anode chamber 102 has an inlet 108 and outlet 110 to permit the flow of water through the anode chamber 100. The cathode chamber 104 similarly has an inlet 112 and outlet 114 to permit the flow of water through the cathode chamber 104. The salt solution chamber 106 has an inlet 116 and outlet 118. The electrolysis cell 100 preferably includes a housing to hold all of the components together.

The anode chamber 102 is separated from the salt solution chamber by an anode electrode 120 and an anion ion exchange membrane 122. The anode electrode 120 may be positioned adjacent to the anode chamber 102 with the membrane 122 located between the anode electrode 120 and the salt solution chamber 106. Alternatively, the membrane 122 may be positioned adjacent to the anode chamber 102 with the anode electrode 120 located between the membrane 122 and the salt solution chamber 106.

The cathode chamber 104 is separated from the salt solution chamber by a cathode electrode 124 and a cathode ion exchange membrane 126. The cathode electrode 124 may be positioned adjacent to the cathode chamber 104 with the membrane 126 located between the cathode electrode 124 and the salt solution chamber 106. Alternatively, the membrane 126 may be positioned adjacent to the cathode chamber 104 with the cathode electrode 124 located between the membrane 126 and the salt solution chamber 106.

The electrodes preferably are constructed of metal to permit a voltage potential to be applied between the anode chamber and cathode chamber. The metal electrodes are generally planar and have similar dimensions and cross-sectional surface area to that of the ion exchange membranes. The electrodes are configured to expose a substantial portion of the surface of the ion exchange members to the water in their respective anode chamber and cathode chamber. This permits the migration of ionic species between the salt solution chamber, anode chamber and cathode chamber. Preferably, the electrodes have a plurality of passages or apertures evenly spaced across the surface of the electrodes.

A source of electrical potential is connected to the anode electrode 120 and cathode electrode 124 so as to induce an oxidation reaction in the anode chamber 102 and a reduction reaction in the cathode chamber 104.

The ion exchange membranes 122 and 126 used in the electrolysis cell 100 may be constructed of any suitable material to permit the exchange of ions between the salt solution chamber 106 and the anode chamber 102 such as, e.g., chloride ions (Cl−) and between the salt solution salt solution chamber 106 and the cathode chamber 104 such as, e.g., sodium ions (Na+). The anode ion exchange membrane 122 and cathode ion exchange membrane 126 may be made of the same or different material of construction. Preferably, the anode ion exchange membrane comprises a fluorinated polymer. Suitable fluorinated polymers include, for example, perfluorosulfonic acid polymers and copolymers such as perfluorosulfonic acid/PTFE copolymers and perfluorosulfonic acid/TFE copolymers. The ion exchange membrane may be constructed of a single layer of material or multiple layers. Suitable ion exchange membrane polymers can include one or more ion exchange membrane polymers marketed under the trademark Nafion.

The source of the water for the anode chamber 102 and cathode chamber 104 of the electrolysis cell 100 may be any suitable water supply. The water may be from a municipal water supply or alternatively pretreated prior to use in the electrolysis cell. Preferably, the water is pretreated and is selected from the group consisting of softened water, purified water, distilled water, and deionized water. More preferably, the pretreated water source is ultrapure water obtained using reverse osmosis purification equipment.

The salt water solution for use in the salt water chamber 106 can include any aqueous salt solution that contains suitable ionic species to produce the ORP water solution. Preferably, the salt water solution is an aqueous sodium chloride (NaCl) salt solution, also commonly referred to as a saline solution. Other suitable salt solutions can include other chloride salts such as potassium chloride, ammonium chloride and magnesium chloride as well as other halogen salts such as potassium and bromine salts. The salt solution can contain a mixture of salts.

The salt solution can have any suitable concentration. For example, the salt solution can be saturated or concentrated. Preferably, the salt solution is a saturated sodium chloride solution.

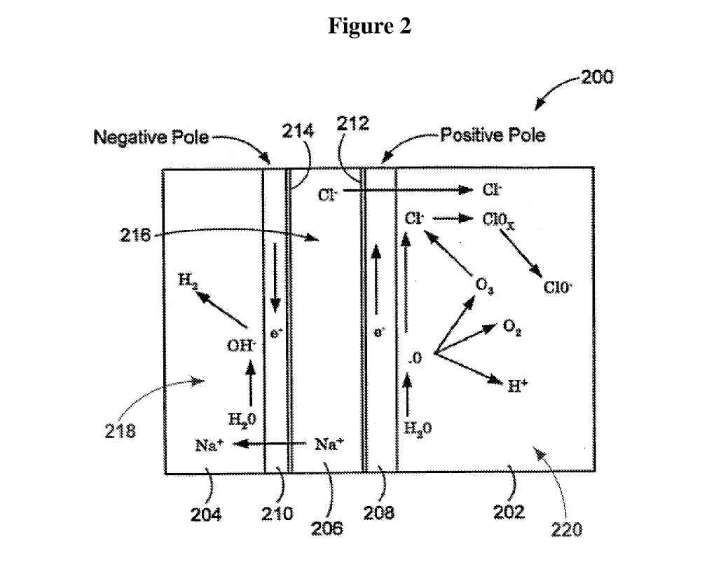

FIG. 2 illustrates what are believed to be various ionic species produced in the three chambered electrolysis cell useful in connection with the invention. The three chambered electrolysis cell 200 includes an anode chamber 202, cathode chamber 204, and a salt solution chamber 206. Upon application of a suitable electrical current to the anode 208 and cathode 210, the ions present in the salt solution flowing through the salt solution chamber 206 migrate through the anode ion exchange membrane 212 and cathode ion exchange membrane 214 into the water flowing through the anode chamber 202 and cathode chamber 204, respectively.

Positive ions migrate from the salt solution 216 flowing through the salt solution chamber 206 to the cathode water 218 flowing through the cathode chamber 204. Negative ions migrate from the salt solution 216 flowing through the salt solution chamber 206 to the anode water 220 flowing through the anode chamber 202.

Preferably, the salt solution 216 is aqueous sodium chloride (NaCl), which contains both sodium ions (Na⁺) and chloride ions (Cl⁻) ions. Positive Na⁺ ions migrate from the salt solution 216 to the cathode water 218. Negative Cl⁻ ions migrate from the salt solution 216 to the anode water 220.

The sodium ions and chloride ions may undergo further reaction in the anode chamber 202 and cathode chamber 204. For example, chloride ions can react with various oxygen ions and other species (e.g., oxygen containing free radicals, O₂, O₃) present in the anode water 220 to produce ClOn− and ClO⁻. Other reactions may also take place in the anode chamber 202 including the formation of oxygen free radicals, hydrogen ions (H⁺), oxygen (e.g., as O₂), ozone (O₃), and peroxides. In the cathode chamber 204, hydrogen gas (H₂), sodium hydroxide (NaOH), hydroxide ions (OH⁻), and other radicals may be formed.

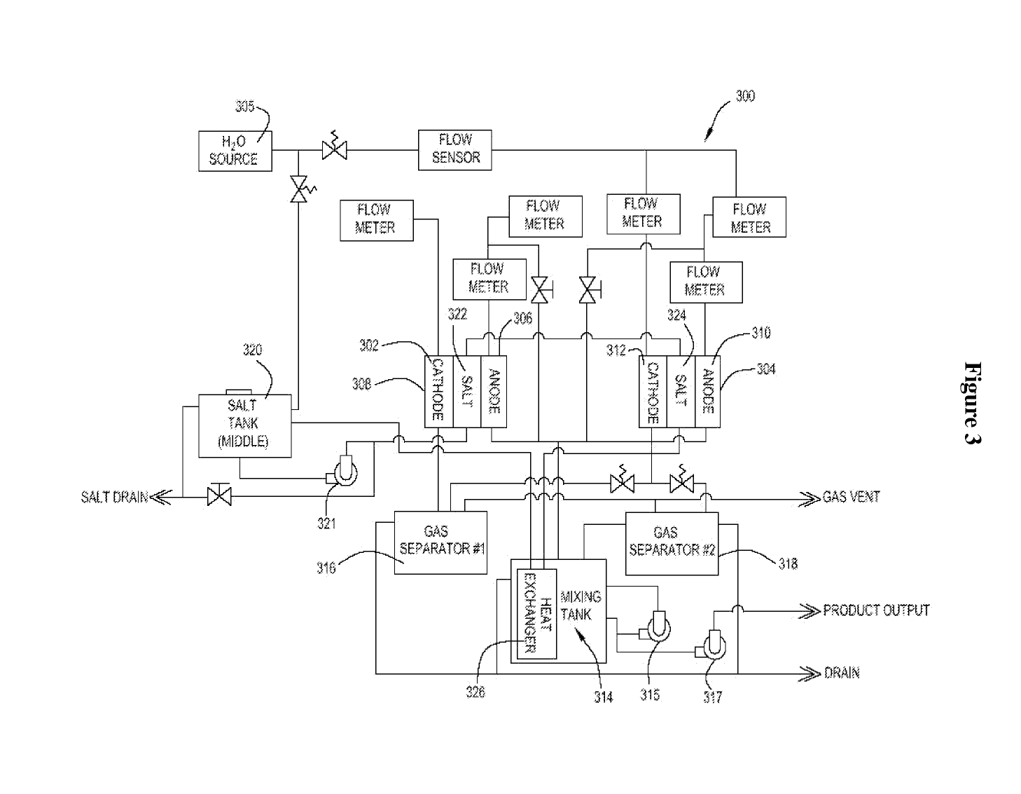

The apparatus for producing the ORP water solution also can be constructed to include at least two three chambered electrolysis cells. Each of the electrolytic cells includes an anode chamber, cathode chamber, and salt solution chamber separating the anode and cathode chambers. The apparatus includes a mixing tank for collecting the anode water produced by the electrolytic cells and a portion of the cathode water produced by one or more of the electrolytic cells. Preferably, the apparatus further includes a salt recirculation system to permit recycling of the salt solution supplied to the salt solution chambers of the electrolytic cells. A diagram of an exemplary process for producing an ORP water solution using two electrolysis cells is shown in FIG. 3.

The process 300 includes two three-chambered electrolytic cells, specifically a first electrolytic cell 302 and second electrolytic cell 304. Water is transferred, pumped or otherwise dispensed from the water source 305 to anode chamber 306 and cathode chamber 308 of the first electrolytic cell 302 and to anode chamber 310 and cathode chamber 312 of the second electrolytic cell 304. Advantageously, this process can produce from about 1 liter/minute to about 50 liters/minute of ORP water solution. The production capacity may be increased by using additional electrolytic cells. For example, three, four, five, six, seven, eight, nine, ten or more three-chambered electrolytic cells may be used to increase the output of the ORP water solution administered in accordance with the invention.

The anode water produced in the anode chamber 306 and anode chamber 310 are collected in the mixing tank 314. A portion of the cathode water produced in the cathode chamber 308 and cathode chamber 312 is collected in mixing tank 314 and combined with the anode water. The remaining portion of cathode water produced in the process is discarded. The cathode water may optionally be subjected to gas separator 316 and/or gas separator 318 prior to addition to the mixing tank 314. The gas separators remove gases such as hydrogen gas that are formed in cathode water during the production process.

The mixing tank 314 may optionally be connected to a recirculation pump 315 to permit homogenous mixing of the anode water and portion of cathode water from electrolysis cells 302 and 304. Further, the mixing tank 314 may optionally include suitable devices for monitoring the level and pH of the ORP water solution. The ORP water solution may be transferred from the mixing tank 314 via pump 317 for application in disinfection or sterilization at or near the location of the mixing tank. Alternatively, the ORP water solution may be dispensed into one or more suitable containers for shipment to a remote site (e.g., warehouse, hospital, etc.).

The process 300 further includes a salt solution recirculation system to provide the salt solution to salt solution chamber 322 of the first electrolytic cell 302 and the salt solution chamber 324 of the second electrolytic cell 304. The salt solution is prepared in the salt tank 320. The salt is transferred via pump 321 to the salt solution chambers 322 and 324. Preferably, the salt solution flows in series through salt solution chamber 322 first followed by salt solution chamber 324. Alternatively, the salt solution may be pumped to both salt solution chambers simultaneously.

Before returning to the salt tank 320, the salt solution may flow through a heat exchanger 326 in the mixing tank 314 to control the temperature of the ORP water solution as needed.

The ions present in the salt solution are depleted over time in the first electrolytic cell 302 and second electrolytic cell 304. An additional source of ions periodically can be added to the mixing tank 320 to replace the ions that are transferred to the anode water and cathode water. The additional source of ions may be used, e.g., to maintain a constant pH of the salt solution, which can to drop (i.e., become acidic) over time. The source of additional ions may be any suitable compound including, for example, salts such as, e.g., sodium chloride. Preferably, sodium hydroxide is added to the mixing tank 320 to replace the sodium ions (Na⁺) that are transferred to the anode water and cathode water.

Following its preparation, the ORP water solution can be transferred to one or more suitable containers, e.g., a sealed container for distribution and sale to end users such as, e.g., health care facilities including, e.g., hospitals, nursing homes, doctor offices, outpatient surgical centers, dental offices, and the like. Suitable containers can include, e.g., a sealed container that maintains the sterility and stability of the ORP water solution held by the container. The container can be constructed of any material that is compatible with the ORP water solution. Preferably, the container is generally non-reactive with one or more ions or other species present in the ORP water solution.

Preferably, the container is constructed of plastic or glass. The plastic can be rigid so that the container is capable of being stored on a shelf. Alternatively, the container can be flexible, e.g., a container made of flexible plastic such as, e.g., a flexible bag.

Suitable plastics can include, e.g., polypropylene, polyester terephthalate (PET), polyolefin, cycloolefin, polycarbonate, ABS resin, polyethylene, polyvinyl chloride, and mixtures thereof. Preferably, the container comprises one or more polyethylenes selected from the group consisting of high-density polyethylene (HDPE), low-density polyethylene (LDPE), and linear low-density polyethylene (LLDPE). Most preferably, the container is constructed of high density polyethylene.

The container preferably has an opening to permit dispensing of the ORP water solution. The container opening can be sealed in any suitable manner. For example, the container can be sealed with a twist-off cap or stopper. Optionally, the opening can be further sealed with a foil layer.

The headspace gas of the sealed container can be air or any other suitable gas, which preferably does not react with one or more species in the ORP water solution. Suitable headspace gases can include, e.g., nitrogen, oxygen, and mixtures thereof.

The following examples further illustrate the invention but, of course, should not be construed as in any way limiting in its scope.

### Materials and Methods Used in Examples 1-2

The following examples demonstrate the antiviral characteristics of an ORP water solution. More specifically, Examples 1 and 2 show inactivation of two strain types of the influenza virus (i.e., H1N1 and H3N1, respectively) when exposed to Microcyn® as in vitro suspensions. The Microcyn® solution (Oculus Innovative Sciences, Petaluma, Calif.) employed is composed of 99.99% water, 0.002% hypochlorous acid, and 0.003% sodium hypochlorite. Microcyn® is pH neutral with a range of 7.2 to 7.8, is non-toxic, and is non-corrosive.

Concentrations representing 1×, 5×, 10×, and 100× normal test dosage were tested on the various cell lines to determine if the Microcyn® would have any toxic affect. This was accomplished by recognizing that the test concentration was used at a dilution of 1:10 in cell culture media, constituting 1×. A 10× solution was 1:1 test Microcyn® to cell culture media. 100× is 10:1 Microcyn® to cell culture media. The different host cells were exposed to these different concentrations of the test Microcyn® for a period of two hours, removed and replaced with 100% cell culture media, and observed for five consecutive days for any signs of cytotoxicity to the host cells as well as retardation of cell proliferation. No toxicity was observed and no reduction in cell proliferation and viability, based on cell counts, was detected.

Influenza A H1N1 [Johannesburg 82/96] (Example 1) and Influenza A H3N1 [Sydney 5/97] (Example 2) were obtained from the California Department of Health Services Viral and Rickettsial Disease Laboratory (VRDL), Richmond, Calif.

To assay both strains of Influenza A viruses, MMU cells were obtained from the VRDL. Tissue cultures were propagated in Minimum Essential Medium with Hank's Salts supplemented with 10% heat-inactivated (56° C. for 30 minutes) Fetal Bovine Serum (FBS), 1% (8.8% w/v) Sodium Bicarbonate, 2% (3% w/v) L-glutamine, 0.5% Pen-Strep (20,000 U/ml), and 0.05% Fungizone (1 mg/ml) for closed system propagation. For the open system test assay, Minimum Essential Medium with Earle's salts was used, FBS was reduced to 5%, and sodium bicarbonate was increased to 2%.

Host cells were planted and grown to confluence in 48-well micro-titer plates several days prior to performance of the test assay. Three 48-well plates were required for each virus assay. One control plate, one 1-minute exposure plate, and one 5-minute exposure plate were used for each virus tested. Prior to inoculation of the plates with any test substance, the existing culture media was removed by aspiration leaving behind approximately 0.1 ml of media to prevent the cell sheet from damage due to drying.

There are 8 rows of 6 wells per row for a total of 48 wells per plate. The first row of all three plates was the cell control. The second row of the control plate began with a dilution of 10-4 and continued through row 8, ending with a ten-fold dilution series from 10-4 to 10-10. Other than the cell control row on the control plate, all other wells received an appropriate dilution of virus in bovine albumin-phosphate buffered saline (BA-PBS) diluent, but untreated with Microcyn®. Each dilution in the series was inoculated into 6 wells equal to one row. For the 1-minute and 5-minute 48-well microtiter test plates, row 1 was the cell control row and each subsequent row represented a dilution in the series beginning with 10-2 and ending with 10-8. The inoculum for the 1-minute and 5-minute plates was composed of an appropriately diluted virus having been exposed to the Microcyn® for either 1 minute or 5 minutes and subsequently inactivated in cell culture media.

Following serial dilutions of test virus in BA-PBS diluent, the 0.2 ml of the challenge viruses at the appropriate dilution were added to 1.8 ml of test Microcyn® and triturated to create a homogenous suspension and a calibrated time was started. At the desired time interval (1 minute and 5 minutes), 0.5 ml of the virus and Microcyn® suspension was transferred to 4.5 ml of cell culture media and gently mixed by trituration to inactivate the virucidal activity of the Microcyn®. A 0.3 ml portion of this suspension was placed on the cells in the 48-well microtiter plates. The plates were then incubated for one hour at 36° C. and 5% CO₂. At the end of the incubation period, an additional 0.5 ml of media was added and plates were returned for incubation. A 0.3 ml portion of the virus suspension without exposure to the Microcyn® was added to the control plate, incubated for the same one hour time interval and then 0.5 ml of media was added and plates returned with others for incubation. The Influenza A viruses do not develop a distinguishable cytopathic effect (CPE), therefore an immunofluorescence technique was utilized to evaluate results. Chamber slides were prepared in parallel for monitoring the rate of infectivity of the test plates throughout the incubation period post-inoculation. Once infectivity of the cells in the chamber slides was detected at a dilution of 10-8, the test plates were removed from incubation and analyzed by fluorescence antibody (FA) testing.

At the conclusion of the assay, results were recorded using a 50% endpoint for visually observable infection through FA to determine the Tissue Culture Infective Dose 50% endpoint (without consideration for the proportionate distance calculations) for the control plate and the Microcyn® test plates. The difference in endpoints between the control plate and test plates represents the degree of reduction of infectious viral particles and is expressed in whole logs.

The Outgrowth and Maintenance Medium for Cell Culture (Open System/Cell Suspension—for 100 ml of medium (1×MEM)) was prepared as follows:

9.5 ml 10× Minimal Essential Medium Eagle with Earle's salts (Sigma brand stock #M0275)
5.0 ml fetal bovine serum (inactivated at 56° C. for 30 min)
2.0 ml 8.8% sodium bicarbonate

### 2.0 ml 3% L-glutamine

0.5 ml penicillin-streptomycin (20,000 U/ml each)
0.05 ml fungizone (1 mg/ml)
QS to 100 ml with sterile distilled water.

The Outgrowth and Maintenance Medium for Cell Culture (Closed System/Cell Passage Media—for 100 ml of medium (1× MEM)) was prepared as follows:

9.0 ml 10× Minimal Essential Medium Eagle with Earle's salts (Sigma brand stock#M0275)
10.0 ml fetal bovine serum (inactivated at 56° C. for 30 min)
1.0 ml 8.8% sodium bicarbonate

### 2.0 ml 3% L-glutamine

0.5 ml penicillin-streptomycin (20,000 U/ml each)
0.05 ml fungizone (1 mg/ml)
QS to 100 ml with sterile distilled water.
Use 40 ml of medium for each T-150 of cells.

Reagent A was prepared as follows:

0.75% Bovine Albumin in PBS pH 7.4 (Use for virus dilutions and serum dilutions. Makes 4 L 1×).
Solution A: Weigh into a 4 L beaker: 32.0 g NaCl, 0.8 g KCl, 0.4 g MgCl₂.6H₂O, 0.3 g CaCl₂.2H₂O. Add 2000 ml Milli-Q water. Stir with stirring bar until dissolved. Weigh and add to the beaker: 30.0 g Bovine Albumin powder (fraction V);
Solution B: Weigh into a 2 L Erlenmeyer flask: 2.44 g Na₂HPO₄, 0.80 g KH2PO₄. Add 1600 ml Milli-Q water. Swirl or stir until dissolved. Add 3.2 m 1% Phenol Red solution. Pour the flask of Solution B into the beaker of Solution A. Continue stirring until dissolved. Check pH with meter using single standard method. Adjust to pH 7.4 with a few ml of 1N NaOH. Bring final volume to 4 L. Membrane filter with 0.2 micrometer pore size to sterilize. Dispense and store at 4° C. Pen-Strep-20,000 U/ml at 0.5% and 0.05% fungizone may be added by user at time of use.

### Example 1

The following tables show inactivation of influenza A H1N1 Johannesburg when exposed to Microcyn® at various times post-infection (as measured by fluorescent antibody testing for infection using Flu A-FITC, see procedure outlined above):

Day 4 post-infection:

Specimen Control Plate

| | Cell Control | 10\hat{ }−4 | 10\hat{ }−5 | 10\hat{ }−6 | 10\hat{ }−7 | 10\hat{ }−8 | 10\hat{ }−9 | 10\hat{ }−10 |
|---|---|---|---|---|---|---|---|---|
| | 1 | 2 | 3 | 4 | 5 | 6 | 7 | 8 |
| A | 0 | + | + | + | + | + | + | + |
| B | 0 | + | + | + | + | + | 0 | + |
| C | 0 | + | + | + | + | + | + | + |
| D | 0 | + | + | + | + | + | + | 0 |
| E | 0 | + | + | + | + | + | + | + |
| F | 0 | + | + | + | + | + | 0 | 0 |

Specimen One Minute Exposure to SOS

| | Cell Control | 10\hat{ }−2 | 10\hat{ }−3 | 10\hat{ }−4 | 10\hat{ }−5 | 10\hat{ }−6 | 10\hat{ }−7 | 10\hat{ }−8 |
|---|---|---|---|---|---|---|---|---|
| | 1 | 2 | 3 | 4 | 5 | 6 | 7 | 8 |
| A | 0 | 0 | 0 | 0 | 0 | 0 | 0 | 0 |
| B | 0 | 0 | 0 | 0 | 0 | 0 | 0 | 0 |
| C | 0 | 0 | 0 | 0 | 0 | 0 | 0 | 0 |
| D | 0 | 0 | 0 | 0 | 0 | 0 | 0 | 0 |
| E | 0 | 0 | 0 | 0 | 0 | 0 | 0 | 0 |
| F | 0 | 0 | 0 | 0 | 0 | 0 | 0 | 0 |

Specimen Five Minute Exposure to SOS

| | Cell Control | 10\hat{ }−2 | 10\hat{ }−3 | 10\hat{ }−4 | 10\hat{ }−5 | 10\hat{ }−6 | 10\hat{ }−7 | 10\hat{ }−8 |
|---|---|---|---|---|---|---|---|---|
| | 1 | 2 | 3 | 4 | 5 | 6 | 7 | 8 |
| A | 0 | 0 | 0 | 0 | 0 | 0 | 0 | 0 |
| B | 0 | 0 | 0 | 0 | 0 | 0 | 0 | 0 |
| C | 0 | 0 | 0 | 0 | 0 | 0 | 0 | 0 |
| D | 0 | 0 | 0 | 0 | 0 | 0 | 0 | 0 |
| E | 0 | 0 | 0 | 0 | 0 | 0 | 0 | 0 |
| F | 0 | 0 | 0 | 0 | 0 | 0 | 0 | 0 |

There was no detectable infection in any of the wells inoculated with Microcyn®-treated H1N1 for either 1 min or 5 min exposures. Control plate demonstrated significant levels of infection.

### Example 2

The following tables show inactivation of influenza A H3N1 Sydney when exposed to Microcyn® at various times post-infection (as measured by fluorescent antibody testing for infection using Flu A-FITC, see procedure outlined above):

Day 4 post-infection:

Specimen Control Plate

| | Cell Control | 10\hat{ }−4 | 10\hat{ }−5 | 10\hat{ }−6 | 10\hat{ }−7 | 10\hat{ }−8 | 10\hat{ }−9 | 10\hat{ }−10 |
|---|---|---|---|---|---|---|---|---|
| | 1 | 2 | 3 | 4 | 5 | 6 | 7 | 8 |
| A | 0 | + | + | + | + | + | + | + |
| B | 0 | + | + | + | + | + | + | 0 |
| C | 0 | + | + | + | + | 0 | + | 0 |
| D | 0 | + | + | + | + | + | 0 | 0 |
| E | 0 | + | + | + | + | 0 | 0 | + |
| F | 0 | + | + | + | + | + | + | 0 |

Specimen One Minute Exposure to Cidalcyn

| | Cell Control | 10\hat{ }−2 | 10\hat{ }−3 | 10\hat{ }−4 | 10\hat{ }−5 | 10\hat{ }−6 | 10\hat{ }−7 | 10\hat{ }−8 |
|---|---|---|---|---|---|---|---|---|
| | 1 | 2 | 3 | 4 | 5 | 6 | 7 | 8 |
| A | 0 | 0 | 0 | 0 | 0 | 0 | 0 | 0 |
| B | 0 | 0 | 0 | 0 | 0 | 0 | 0 | 0 |
| C | 0 | 0 | 0 | 0 | 0 | 0 | 0 | 0 |
| D | 0 | 0 | 0 | 0 | 0 | 0 | 0 | 0 |
| E | 0 | 0 | 0 | 0 | 0 | 0 | 0 | 0 |
| F | 0 | 0 | 0 | 0 | 0 | 0 | 0 | 0 |

Specimen Five Minute Exposure to Cidalcyn

| | Cell Control | 10\hat{ }−2 | 10\hat{ }−3 | 10\hat{ }−4 | 10\hat{ }−5 | 10\hat{ }−6 | 10\hat{ }−7 | 10\hat{ }−8 |
|---|---|---|---|---|---|---|---|---|
| | 1 | 2 | 3 | 4 | 5 | 6 | 7 | 8 |
| A | 0 | 0 | 0 | 0 | 0 | 0 | 0 | 0 |
| B | 0 | 0 | 0 | 0 | 0 | 0 | 0 | 0 |
| C | 0 | 0 | 0 | 0 | 0 | 0 | 0 | 0 |
| D | 0 | 0 | 0 | 0 | 0 | 0 | 0 | 0 |
| E | 0 | 0 | 0 | 0 | 0 | 0 | 0 | 0 |
| F | 0 | 0 | 0 | 0 | 0 | 0 | 0 | 0 |

There was no detectable infection in any of the wells inoculated with Microcyn®-treated H3N1 for either 1 min or 5 min exposures. Control plate demonstrated significant levels of infection.

Examples 1 and 2 (i.e., the antiviral efficacy of Microcyn® solution against both influenza A strains H1N1 and H3N1, respectively) demonstrate log 10 reductions at least about 7 and are summarized in the table below:

| | Log Reduction after | Log Reduction after |
|---|---|---|
| Virus Type | One Minute Exposure | Five Minute Exposure |
| H1N1 | ≧7 logs | ≧7 logs |
| H3N1 | ≧7 logs | ≧7 logs |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments can become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating, reducing, or preventing the incidence of a viral infection in a patient comprising administering a therapeutically effective amount of an oxidative reductive potential (ORP) water solution to an infectious virus present on the surface of a tissue, wherein the infectious virus is an influenza virus and the tissue is selected from the group consisting of lower and upper respiratory tract tissue, corneal tissue, inner ear tissue, urinary tract tissue, mucosa tissue, dental tissue and synovial tissue.

2. The method of claim 1, wherein the pH of the ORP water solution is from about 6.0 to about 8.0.

3. The method of claim 1, wherein the ORP water solution comprises at least one free chlorine species selected from the group consisting of hypochlorous acid, hypochlorite ions, sodium hypochlorite, chlorite ions, chloride ions, dissolved chlorine gas, hydrogen peroxide, chlorine dioxide, and combinations thereof.

4. The method of claim 3, wherein the concentration of free chlorine species in the ORP water solution is from about 10 ppm to about 400 ppm.

5. The method of claim 1, wherein the ORP water solution comprises a mixture of cathode water and anode water.

6. The method of claim 1, wherein the ORP water solution has a potential between about −400 mV and about +1300 mV.

7. The method of claim 1, wherein the ORP water solution is stable for at least about two months.

8. The method of claim 1, wherein the influenza virus is an Influenza A virus selected from the group consisting essentially of subtype H1N1 or subtype H3N1.

9. The method of claim 1, wherein the patient is a human.

10. The method of claim 1, wherein the ORP water solution is administered using an endoscope.

11. The method of claim 1, wherein the ORP water solution is administered as a lavage, drop, rinse, spray, mist, aerosol, steam or combination thereof.

12. The method of claim 1, wherein the ORP water solution is administered in the form of droplets having a diameter in the range of from about 0.1 micron to about 100 microns.

13. A method of reducing or preventing the incidence of a viral infection in a patient associated with a medical device comprising contacting the medical device with an effective amount of an oxidative reductive potential (ORP) water solution, wherein an influenza virus is present on the surface of the device.

14. The method of claim 13, wherein the pH of the ORP water solution is from about 6.0 to about 8.0.

15. The method of claim 13, wherein the ORP water solution comprises at least one free chlorine species selected from the group consisting of hypochlorous acid, hypochlorite ions, sodium hypochlorite, chlorite ions, chloride ions, dissolved chlorine gas, hydrogen peroxide, chlorine dioxide, and combinations thereof.

16. The method of claim 15, wherein the concentration of free chlorine species in the ORP water solution is from about 10 ppm to about 400 ppm.

17. The method of claim 13, wherein the medical device is selected from a group consisting of epicardial leads, cardiac and cerebral pacemakers, defibrillators, left ventricular assist devices, mechanical heart valves, total artificial hearts, ventriculoatrial shunts, pledgets, patent ductus arteriosus occlusion devices, atrial septal defect and ventricular septal defect closure devices, conduits, patches, peripheral vascular stents, coronary artery stents, vascular grafts, abdominal mesh reinforcements, hemodialysis shunts, intra-aortic balloon pumps, angioplasty balloon catheters, angiography catheters, vena caval filters, endotracheal tubes, cochlear implants, tympanostomy tubes, bioabsorbable osteoconductive drug-releasing hard tissue fixation devices, artificial joint replacements and other orthopedic implants, contact lenses, intrauterine devices, dental and orthodontic appliances and fixtures, urinary catheters, intravenous catheters, sutures and surgical staples.

18. The method of claim 13, wherein the concentration of the influenza virus present on the surface of the device is reduced by at least about five logs (10⁵) within two minutes of exposure of the influenza virus to the ORP water solution.

19. The method of claim 18, wherein the concentration of the influenza virus present on the surface of the device is reduced by at least about seven logs (10⁷) within one minute of exposure of the influenza virus to the ORP water solution.
